(19) United States
(12) Patent Application Publication
(10) Pub. No.: US 2006/0137111 A1
(43) Pub. Date: Jun. 29, 2006

(54) One step hair coloring using salts

(75) Inventors: Van Au, Naperville, IL (US); Jitendra Patel, Fox River Grove, IL (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 11/025,295

(22) Filed: Dec. 29, 2004

(51) Int. Cl. A61K 8/49; A61K 8/22

(52) U.S. Cl. 8/406

(57) ABSTRACT

A permanent hair coloring composition is described, the hair coloring composition being produced by combining individually packaged dye, developer and salt components just prior to application to the hair, wherein the dye, developer and salt components are formulated to provide the hair coloring composition with relatively high levels of ammonium carbonate salt, additional soluble carbonate salt other than ammonium carbonate salt, and chelating agent, the ammonium carbonate salt to additional carbonate salt being present in a ratio, by weight of from about 1:0.6 to about 1:1.6, wherein, based on the combined weight of the dye, developer and salt components, the composition has a total soluble carbonate salt content of greater than about 10% by weight, and at least about 1 % by weight of chelating agent. Kits for preparing and methods for using such hair coloring compositions are also disclosed.

### FIELD OF THE INVENTION

This invention relates to methods and compositions for providing more vibrant, long-lasting color to hair while also achieving less damaging bleaching and greater lightening or lift. The invention also relates to kits from which such compositions are formed.

### BACKGROUND OF THE INVENTION

To color human or animal hair using conventional oxidative dye technology, a mixture of suitable oxidative coloring agents and at least one oxidizing agent or developer, typically hydrogen peroxide, is commonly employed. Hair colorants based on oxidative dyes generally have two parts, i.e., a dye component and a developer component, each of which is ordinarily provided in a liquid or other fluid form, e.g., gel. Just before use, the dye and developer components are mixed together to form the colorant composition. The kits in which these components are provided include dye systems known as “box colorants”, which are generally formulated as single-use products. Box colorants include kits for home as well as salon use.

Carbonates are among the ingredients that have been disclosed for use in hair colorant compositions based on oxidative dyes. For example, carbonates have been disclosed for use as buffers or pH adjusters. As a buffer or pH adjuster, carbonates are used in relatively small amounts, typically not exceeding 1% by weight of the colorant composition after mixing. The use of carbonates has also been linked to improving lightening or lift.

The use of carbonates in hair colorant compositions is disclosed in publications that include the following:

U.S. Pat. No. 5,131,912 discloses durable 2-part hair dyeing agents composed of a first agent comprising as essential components at least one compound that forms HCO₃⁻ by dissociation in water, an alkali generating substantially no irritating odor, and a dye for hair and having a pH of 8.12 to 9.0, and a second agent comprising hydrogen peroxide and a buffer solution and having a pH of 2.0 to 4.0, the weight ratio of the first agent and the second agent to be mixed being such that the pH of the mixture of the two is in a range of from 6.5 to 7.9. The patent discloses that these 2-part hair dyeing agents require only a short dyeing time, create little damage to hair, have no irritating or disagreeable odor and have high dyeing effect.

U.S. Pat. No. 5,525,123 discloses a hair dyeing composition based on oxidation dyestuff precursors which dyes and brightens the hair, containing, besides the at least one developing and at least one coupling agent, at least one metal salt and at least one ammonium compound selected from the group of ammonium chloride, ammonium sulfate, ammonium carbonate, ammonium bicarbonate, and ammonium carbamate.

U.S. Patent Application Publication No. 2004/0019980 discloses a hair coloring composition comprising two compositions which are mixed just prior to application to the hair:
(a) a composition comprising a water-soluble peroxygen oxidizing agent; and
(b) a composition comprising one or more oxidative hair coloring agents selected from the group consisting of an aromatic diamine, an amino phenol, a naphthol, a polyhydric phenol, a catechol and mixtures thereof;

wherein the composition comprising one or more oxidative hair coloring agents further comprises at least one water soluble carbonate releasing salt; and optionally a water soluble ammonium salt. In Example 1, hair coloring compositions having 1.0-5.0% by weight of water soluble carbonate releasing salt and 0-5.0% by weight of water soluble ammonium salt are disclosed.

U.S. Pat. No. 6,743,264 discloses a method for achieving permanent hair color changes, which method comprises the steps of:
1) contacting the hair with a substantially inactive mixture of oxidative dye precursors (the hair colorant part) at a pH of about 8 or about 7 or below for a period of about 30 seconds to about 60 minutes; and
2) contacting the hair with an oxidizing compound (the hair color developer part) at a pH of about 8 to about 11 for a period of about 30 seconds to about 60 minutes; and
3) rinsing the hair with water.
   The patent further discloses that compounds that form HCO₃⁻ by dissociating in water (referred to as “ion forming compounds”) may be present in the hair colorant and hair color developer parts to maintain a desired pH level. Identified as non-limiting examples of suitable ion forming compounds are: Na₂CO₃, NaHCO₃, K₂CO₃, (NH₄)₂CO₃, NH₄HCO₃, CaCO₃, and Ca(HCO₃)₂, and mixtures thereof.

U.S. Patent Application No. 2002/0189034 discloses a two-agent hair dyeing/bleaching composition comprising a primary agent containing an alkali agent and a secondary agent containing an oxidizing agent, wherein the primary agent contains: (a) at least one alkali agent selected from the group consisting of ammonia water and monoethanolamine, (b) at least one water-soluble ammonium salt selected from the group consisting of ammonium carbonate and ammonium hydrogencarbonate, and (c) at least one first pH adjuster selected from the group consisting of polycarboxylic acids and their salts. At paragraph 0026, it is disclosed that that the primary agent “preferably contains the alkali agent of component (a) at 1-25 wt % (where the ammonia water weight is expressed in terms of ammonia water at a concentration of 28 wt %), the water-soluble ammonium salt of component (b) at 0.5-20wt % and the first pH adjuster component (c) at 0.1-10 wt %.” At paragraph 0043 the application notes that the composition may additionally contain a hair dye when it is to be used as a “dyeing agent”.

U.S. Patent Application Publication No. 2004/0010865 discloses hair dye compositions having a pH of from 8.5-12 which comprise (A) ammonia or an ammonium salt, (B) a carbonate (other than an ammonium salt), (C) a transition metal salt, and (D) a chelating agent. The content of ingredients (A) and (B) in the composition are given as 0.01 to 3 mol/kg and 0.001 to 1 mol/kg, respectively. It is further disclosed that the compositions “do not give off an intensely irritating odor and have low irritating property, can change hair into a lighter tone in a short time or can dye hair well in a color ranging from a light color to a deep color, and moreover, assure good retention of the thus-obtained tone or color”.

U.S. Patent Application Publication No. 2004/0098814 discloses a method for the gradual permanent coloring of hair through the use of daily hair care compositions. In the method therein described, the hair is contacted for a period of about 5 seconds to about 5 minutes with a recently prepared mixture of compositions referred to as Parts A and B. Part A is a dye composition in a shampoo or conditioner base at alkaline pH and Part B is a peroxide such as hydrogen peroxide in a conditioner or shampoo base at acidic pH. More particularly, Part A is a mixture of (I) a dye intermediate in a shampoo or conditioner base and a salt component (II) as therein more particularly described. The salt component II is a water soluble ammonium carbonate or carbamate salt at about 0.1% to about 15%, more preferably about 1% to about 10% or a combination of i) a water soluble carbonate releasing salt at about 0.1% to about 15%, more preferably about 1% to about 10% and ii) a water soluble ammonium salt at about 0.1% to about 15%, more preferably about 1% to about 10%.

U.S. Patent Application Publication No. 2004/0098816 discloses a method for the gradual permanent coloring of hair with longer lasting conditioning and with minimized hair damage through the use of daily hair care compositions. In the method therein described, hair is subjected to a number of treatments, having a set time interval between each two consecutive treatments, wherein each treatment comprises:

a) contacting the hair with a recently prepared mixture of a colorant composition comprising:
A) an alkaline dye composition comprising:
   i) an effective amount to color hair of at least one dye intermediate;
   ii) from about 0.1 to about 25% by weight based on the colorant composition of a water soluble ammonium carbonate or carbamate salt;
   iii) from 1 to 5% by weight based on the colorant composition of a chelant; and
   iv) a cosmetically acceptable carrier; and
B) an oxidizing composition comprising:
   i) from 0.1 to 15% by weight based on the coloring composition of a peroxide compound; and
   ii) a cosmetically acceptable carrier; and

b) rinsing the mixture from the hair with water.

Owing to the instability of peroxide in the presence of base, the developer component of peroxide-based hair colorant systems or kits is normally formulated to an acidic pH. At acidic pHs, carbonates are susceptible to decomposition. Accordingly, when present, carbonates are generally formulated as part of the colorant system's dye component or “tint”, rather than its developer component. To minimize weight and the cost of additional ingredients needed to compensate for dilution effects, the amount of solvent provided to the dye component is generally kept relatively low. Solubility limitations and other formulation constraints limit the amount of carbonate that can be contained in the dye component of colorant composition systems. At levels in excess of about 6 to 8 percent by weight, carbonates tend to precipitate or “salt out” of the developer component and can give rise to other stability issues, e.g., breaking of emulsions, undesirable viscosity changes, and the like.

Carbonates degrade peroxide to its more reactive percarbonic and percarbamic species. In addition to the practical limitations as to the amount of carbonate that can be physically incorporated into the fluid components of colorant system kits, the use of high levels of carbonates can give rise to undesirably rapid degradation of peroxide, loss of bleaching efficiency and unacceptable amounts of hair damage. Compensating for these degradation effects by increasing the peroxide level of the colorant composition does not provide a suitable solution, as the use of too much peroxide can be damaging to the hair. Additionally, carbonates can activate the metal ions present on the hair resulting in the formation of undesirable color bodies and hair color that does not appear “natural”.

Hair coloring compositions and methods that provide improved lightening or lift while also minimizing damage to the hair by the coloring process are desired.

### SUMMARY OF THE INVENTION

It has been found that the use of relatively high levels of at least one ammonium carbonate salt in combination with relatively high levels of at least one additional soluble carbonate salt other than an ammonium carbonate salt, and a relatively high level of chelating agent provides colorant compositions having superior lift and dyeing properties. Despite their high level of bleaching efficacy, the subject compositions leave the hair feeling soft and manageable, and provide a conditioning benefit that is particularly surprising in view of the high degree of lightening that these compositions can attain.

In one embodiment of the subject invention there is provided a permanent hair coloring composition comprising the following individual components which are combined just prior to application to the hair:
(a) a dye component comprising oxidation dye precursor,
(b) a developer component comprising peroxide-releasing compound, and
(c) a salt component comprising carbonate-releasing salt,

wherein the dye, developer and salt components are formulated to provide the hair coloring composition with: (i) at least one ammonium carbonate salt and at least one additional soluble carbonate salt other than an ammonium carbonate salt, wherein the ratio, by weight, of ammonium carbonate salt to additional soluble carbonate salt is from about 1:0.6 to about 1:1.6, (ii) a total soluble carbonate salt content of greater than about 10% by weight, based on the combined weight of the dye, developer and salt components, and (iii) at least about 1% by weight of chelating agent, based on the combined weight of the dye, developer and salt components.

In a further embodiment of this invention there is provided a method for coloring hair using the hair coloring composition of the subject invention comprising the steps of:
1) mixing individually packaged dye, developer and salt components to form a hair coloring composition,
2) applying the hair coloring composition to the hair,
3) allowing the hair coloring composition to remain on the hair for a period sufficient to achieve a desired degree of coloration, and
4) rinsing the hair.

In yet another embodiment of this invention there is provided a kit comprising the following individually packaged components:
(a) a dye component comprising oxidation dye precursor,
(b) a developer component comprising peroxide-releasing compound, and
(c) a salt component comprising carbonate-releasing salt,
   wherein the kit further comprises directions instructing that the dye, developer, and salt components are combined just prior to use to form a hair coloring composition, and wherein said components are formulated to provide the resulting hair coloring composition with: (i) at least one ammonium carbonate salt and at least one additional soluble carbonate salt other than an ammonium carbonate salt, wherein the ratio, by weight, of ammonium carbonate salt to additional soluble carbonate salt is from about 1:0.6 to about 1:1.6, (ii) a total soluble carbonate salt content of greater than about 10% by weight, based on the combined weight of the dye, developer and salt components, and (iii) at least about I % by weight of chelating agent, based on the combined weight of the dye, developer and salt components.

### DETAILED DESCRIPTION OF THE INVENTION

The majority of the bleaching reaction is believed to take place within the color bodies or melanosomes of the hair fiber. If active bleaching species, e.g., percarbonates and percarbamates are primarily generated outside, rather than inside of the melanosomes, bleaching efficacy can be low, as a significant portion of the active bleaching species can degrade before diffusing into the melanosomes. Additionally, generating the active bleaching species outside the melanosomes increases the potential for hair damage. Without wishing to be bound to theory, it is believed that the combination of ammonium carbonate, additional soluble carbonate and chelating agent described by the subject invention improves lightening efficacy by controlling the degradation of hydrogen peroxide and increasing the amount of active bleaching species that is generated within the melanosomes and available for the bleaching reaction. It has been found that using the coloring compositions of this invention may provide as much as 4 or more tones of hair lightening without impacting formulation stability. Moreover, despite the high level of carbonate, the coloring compositions of this invention may result in significantly less hair damage than many commercially available hair coloring products.

Throughout the specification and claims, soluble carbonate salts other than ammonium carbonate salts are referred to by the equivalent terms “additional soluble carbonate salt”, “additional carbonate salt”, or simply “additional carbonate”. As used herein, the term “carbonate-releasing salt” refers to soluble carbonate salts as well as compounds and combinations of compounds that release soluble carbonate upon metathesis; the term “carbonate salt” includes not only inorganic and organic carbonates, but also their corresponding bicarbonate, and carbamates. For example, carbonate-releasing salts that provide ammonium carbonate salts include, but are not limited to: ammonium carbonate, ammonium bicarbonate, and ammonium carbamate, with ammonium carbonate being of particular interest in the practice of this invention. Examples of carbonate-releasing salts that provide additional soluble carbonate salts include, but are not limited to: alkali and alkaline earth metal carbonates and bicarbonates, guanidine carbonate, and the like, with the use of alkali metal carbonates, especially sodium carbonate, being of particular interest in the practice of this invention.

The hair coloring compositions of this invention are formulated to contain a total amount of soluble carbonate salt, i.e., ammonium carbonate salt and additional carbonate salt combined, of greater than about 10% by weight, based on the combined weight of the dye, developer and salt components. Preferably, the total amount of soluble carbonate salt is at least about 12% by weight, based on the combined weight of the dye, developer and salt components and, more preferably, from about 12% to about 25% by weight, based on the combined weight of the dye, developer and salt components. In an embodiment of particular interest, the total amount of soluble carbonate salt is from about 13% to about 20% by weight, based on the combined weight of the dye, developer and salt components of the hair coloring composition.

In the practice of this invention the ratio of ammonium carbonate to additional soluble carbonate other than an ammonium-containing carbonate is from about 1:0.6 to about 1:1.6. Without wishing to be bound to theory, it is believed that the ammonium-containing carbonate functions, in part, to solubilize melanine and promote diffusion of the active bleaching species into the melanosomes, while the additional soluble carbonate functions to provide a sufficiently basic pH to promote formation of the active bleaching species and to minimize oxidative damage to the hair. Ratios of preference will depend upon the particular carbonates selected. Ratios of ammonium carbonate salt to additional soluble carbonate salt of from about 1.0:0.7 to about 1.0:1.4, more particularly, from about 1.0:1.0 to about 1.0:1.2, are of particular interest in at least one embodiment of this invention.

In the practice of this invention a separate salt component provides a means of increasing the content of carbonate-releasing salt available to the resulting hair colorant composition. Owing to the solubility limitations of the dye component emulsion, a substantial amount, if not all, of the carbonate-releasing salt present in the colorant compositions of this invention will be provided by the salt component (c). It is, however, also contemplated that a portion of the carbonate-releasing salt may be provided by the dye component (a).

The salt component (c) is desirably provided in solid, as opposed to fluid, form to avoid having to compensate for the dilution effect of additional fluids. It is preferable for the salt component to be provided in particulate form, for example, in powder, particle, granule, pellet, or other solid form that may be readily dissolved in the colorant composition formed by mixing of the dye, developer and salt components. In addition to carbonate-releasing salt, the salt component (c) may further comprise other composition ingredients that may be conveniently incorporated as part of this component, for example, other materials whose solubility in the dye or developer components is limited or that are more conveniently added as a separate, preferably particulate, component. When added in particulate form, the salt component may further comprise filler, binder, and other non-reactive materials that provide volume and/or assist in the production of a component that consumers can easily combine with the dye and developer components of the subject compositions.

Oxidation Dyes

The colorant compositions of this invention comprise one or more oxidation dyes that upon reaction with a peroxide compound such as, for example, hydrogen peroxide, form materials capable of delivering color to the hair. Suitable dyes include dye intermediates or precursors. Precursors known as “primary intermediates” produce colors when oxidized. Another class of precursors, known as “couplers” or “secondary intermediates”, form reactive dye species when oxidized in the presence of a primary intermediate but, in general, do not produce any color when oxidized alone. The coupler is utilized to expand the color range by reaction with the primary intermediate, and may also be used to accelerate color formation. Oxidation dye precursors (primary intermediates and couplers) are described, for example, in Sagarin, “Cosmetic Science and Technology”, Interscience, Special Edition, Volume 2, pages 308 to 310; and “The Chemistry of Synthetic Dyes”, Volume 5, Academic Press, Inc., New York and London (1971).

Non-limiting examples of precursors suitable for use herein and which may function as primary intermediates are the following: 1,4-diamino-benzene (p-phenylenediamine); 1,4-diamino-2-methyl-benzene (p-toluylenediamine); 1,4-diamino-2,6-dimethyl-benzene; 1,4-diamino-3,5-diethyl-benzene; 1,4-diamino-2,5-dimethyl-benzene; 1,4-diamino-2,3-dimethylbenzene; 2-chloro-1,4-diaminobenzene; 1,4-diamino-2-(thiophen-2-yl)benzene; 1,4-diamino-2-(thiophen-3-yl)benzene; 1,4-diamino-2-(pyridin-3-yl)benzene; 2,5-diaminobiphenyl; 1,4-diamino-2-methoxymethyl-benzene; 1,4-diamino-2-aminomethylbenzene; 1,4-diamino-2-hydroxymethyl-benzene; 1,4-diamino-2-(2-hydroxyethoxy)benzene; 2-(2-(acetylamino)ethoxy)-1,4-diaminobenzene; 4-phenylamino-aniline; 4-dimethylamino-aniline; 4-diethylamino-aniline; 4-dipropylamino-aniline; 4-[ethyl(2-hydroxyethyl)amino]-aniline; 4-[di(2-hydroxyethyl)amino]-aniline; 4-[di(2-hydroxyethyl)amino]-2-methyl-aniline; 4-[(2-methoxyethyl)amino]-aniline; 4-[(3-hydroxyropyl)amino]-aniline; 4-[(2,3-dihydroxypropyl)amino-aniline; 1,4-diamino-2-(2-hydroxyethyl)-benzene; 1,4-diamino-2-(1-methylethyl)-benzene; 1,3-bis[(4-aminophenyl)(2-hydroxyethyl)amino]-2-propanol; 1,4-bis[(4-aminophenyl)amino]-butane; 1,8-bis(2,5-diaminophenoxy)-3,6-dioxaoctane; 4-amino-phenol; 4-amino-3-methyl-phenol; 4-amino-3-(hydroxymethyl)-phenol; 4-amino-3-fluoro-phenol; 4-methylamino-phenol; 4-amino-2-(aminomethyl)-phenol; 4-amino-2-(hydroxymethyl)-phenol; 4-amino-2-fluorophenol; 4-amino-2-[(2-hydroxyethyl)-amino]methylphenol; 4-amino-2-methyl-phenol; 4-amino-2-(methoxymethyl)-phenol; 4-amino-2-(2-hydroxyethyl)-phenol; 5-amino-salicylic acid; 2,5-diamino-pyridine; 2,4,5,6-tetraamino-pyrimidine; 4,5-diamino-1-1(2-hydroxyethyl)-1H-pyrazole; 4,5-diamino-1-(1-methylethyl)-1H-pyrazole; 4,5-diamino-1-[(4-methylphenyl)methyl]-1H-pyrazole; 1-[(4-chlorophenyl)methyl]-4,5-diamino-1H-pyrazole; 4,5-diamino-1-methyl-1H-pyrazole; 2-aminophenol; 2-amino-6-methylphenol; and 2-amino-5-methylphenol.

Non-limiting examples of couplers suitable for use herein are the following: N-(3-dimethylamino-phenyl)-urea; 2,6-diamino-pyridine; 2-amino-4-[(2-hydroxyethyl)amino]anisole; 2,4-diamino-1-fluoro-5-methylbenzene; 2,4-diamino-1-methoxy-5-methylbenzene 2,4-diamino-1-ethoxy-5-methyl-benzene; 2,4-diamino-1-(2-hydroxyethoxy)-5-methylbenzene; 2,4-di[(2-hydroxyethyl)amino]-1,5-dimethoxybenzene; 2,3-diamino-6-methoxy-pyridine; 3-amino-6-methoxy-2-(methylamino)pyridine; 2,6-diamino-3,5-dimethoxypyridine; 3,5-diamino-2,6-dimethoxy-pyridine; 1,3-diaminobenzene; 2,4-diamino-1-(2-hydroxyethoxy)benzene; 1,3-diamino4-(2,3-hydroxpropoxy)benzene; 2,4-diamino-1,5-di(2-hydroxyethoxy)-benzene; 1-(2-aminoethoxy)-2,4-diaminobenzene; 2-amino-1-(2-hydroxyethoxy)-4-methylaminobenzene; 2,4-diaminophenoxyacetic acid ester; 3-[di(2-hydroxyethyl)amino]aniline; 4-amino-2-di[(2-hydroxyethyl)amino]-1-ethoxy-benzene; 5-methyl-2-(1-methyethyl)phenol; 3-[(2-hydroxyethyl)amino]aniline; 3-[(2-aminoethyl)amino]aniline; 1,3-di(2,4-diaminophenoxy)propane; di(2,4-diaminophenoxy)methane; 1,3-diamino-2,4-dimethoxybenzene; 2,6-bis(2-hydroxyethyl)aminotoluene; 4-hydroxyindole; 3-dimethylaminophenol; 3-diethylaminophenol; 5-amino-2-methylphenol; 5-amino-4-fluoro-2-methyl-phenol; 5-amino-4-methoxy-2-methylphenol; 5-amino-4-ethoxy-2-methylphenol; 3-amino-2,4-dichlorophenol; 5-amino-2,4-dichlorophenol; 3-amino-2-methyl-phenol; 3-amino-2-chloro-6-methylphenol; 3-aminophenol; 2-[(3-hydroxyphenol)-amino]acetamide; 5-[(2-hydroxyethyl)amino]-4-methoxy-2-methylphenol; 5-[(2-hydroxyethyl)amino]-2-methylphenol; 3-[(2-hydroxyethyl)amino]-phenol; 3-[(2-methoxyethyl)amino]-phenol; 5-amino-2-ethyl-phenol; 5-amino-2-methoxyphenol; 2-(4-amino-2-hydroxyphenoxy)ethanol; 5-[(3-hydroxypropyl)amino]-2-methylphenol; 3-[(2,3-dihydroxypropyl)amino]-2-methylphenol; 3-[(2-hydroxyethyl)amino]-2methylphenol; 2-amino-3-hydroxypyridine; 5-amino-4-chloro-2-methylphenol; 1-naphthol; 2-methyl-1-naphthol; 1,5-dihydroxynaphthalene; 1,7-dihydroxy-naphthalene; 2,3-dihydroxynaphthalene, 2,7-dihydroxy-naphthalene; 2-methyl-1-naphthol-acetate; 1,3-dihydroxybenzene; 1-chloro-2,4-dihydroxy-benzene; 2-chloro-1,3-dihydroxybenzene; 1,2-dichloro-2,4-dihydroxy-4-methylbenzene; 1,5-dichloro-2,4-dihydroxy-benzene; 1,3-dihydroxy-2-ethyl-benzene; 3,4-methylenedioxy-phenol; 3,4-methylenedioxy-aniline; 6-bromo-1-hydroxy-3,4-methylenedioxybenzene; 3,4-diaminobenzoic acid; 3,4-dihydroxy-6-hydroxy-1,4(2H)benzoxazine; 6-amino-3,4-dihydro-1,4(2H)-benzoxazine; 3-methyl-1-phenyl-5-pyrazolone; 5,6-dihydroxyindole; 5,6-dihydroxyindoline; 5-hydroxyindole; and 6-hydroxyindole.

The descriptions of primary intermediates and couplers given above is meant implicitly to include the salt forms of those dye molecules that form stable salts.

The oxidation dye generally comprises from about 0.01 to about 20 percent by weight, more particularly from about 0.1 to about 15 percent by weight of the dye component. In at least one embodiment of interest, the oxidation dye comprises from about 0.1 to about 10 percent by weight of the dye component. In another embodiment of interest, the oxidation dye comprises from about 0.1 to about 5 percent by weight of the dye component.

It should be understood that the precursors described above are by way of example only and are not intended to be exhaustive of oxidative dyes suitable for use herein. Additionally, the hair coloring compositions of this in invention may optionally include non-oxidative and other dye materials. Optional non-oxidative and other dyes suitable for use here include semi-permanent, temporary and other dyes. Non-oxidative 10 dyes as defined herein in include the so-called “direct action dyes”, metallic dyes, metal chelate dyes, fiber reactive dyes and other synthetic and natural dyes. Various types of non-oxidative dyes are described, for example, in “The Science of Hair Care”, edited by C. Zviak, Chapter 7 (pp. 235-261); and “Hair Dyes”, J. C. Johnson, Noyes Data Corp., Park Ridge, U.S.A. (1973) (pp. 3-91 and 113-139).

To prevent premature oxidation, it is typical for the dye component to further comprise one or more antioxidants. Desirably, the dye component is formulated to a pH of from about 9 to about 11, more particularly, from about 9.5 to about 10. Optionally, one or more buffering agents may be included within the dye component to assist in maintaining a desired pH.

Carrier

The dye component further comprises, as a carrier therefor, water and at least one additional cosmetically acceptable solvent or diluent. Generally, the additional solvent or diluent is selected to be miscible with water. Included among the suitable solvents or diluents are mono- and polyhydric alcohols and their ethers, with the C₁-C₁₀ alcohols and ethers thereof and, in at least one embodiment of this invention, the C₂-C₆ alcohols and ethers thereof, being of particular interest. In one embodiment, preferred carriers for the dye component include water and at least one additional solvent selected from the group consisting of ethanol, ethylene glycol, propanol, isopropanol, glycerol, propylene glycol, butanol, and ethylene glycol monoethyl ether.

The carrier for the developer component comprises water, and, optionally, one or more cosmetically acceptable solvents or diluents, for example the cosmetically acceptable alcohols and ethers described above, provided that such solvents and diluents are miscible with water and do not undesirably react with the other materials present in the developer component.

Surfactants

Desirably, the colorant compositions of the subject compositions will further comprise one or more surfactants. Surfactants aid in the uniform distribution of the colorant composition on the hair surface, and assist the user in rinsing the colorant composition from the hair subsequent to treatment. A particularly suitable class of surfactants for use herein is cationic surfactants.

One type of preferred cationic surfactant is amine based and includes alkyl amines, alkylethoxy amines, ethoxylated alkyl amines and alkyl alkanol amines. Preferred alkyl groups have 1 to about 22 carbon atoms and can have a mixture of chainlengths, e.g., methyl and hexadecyl. The term “amines” includes primary, secondary, tertiary and quaternary amines.

A second type of preferred cationic surfactant is amidoamines and includes C₁₂-C₂₂ alkyl or alkylethoxy mono, di and higher (poly)amidoamines which can be ethoxylated or unethoxylated. Nonlimiting examples of such cationic surfactants are: sodium dimethylaminopropyl coco-aspartamide, cocoamidopropyl dimethylamine, olivamidopropyl dimethylamine, soyamidopropyl dimethylamine, tallowamidopropyl dimethylamine, and stearamidoethyl dimethylamine.

Another preferred class of surfactant that is suitable for use in the practice of this invention is nonionic surfactants. This class includes long chain (C₁₂-C₂₂) fatty alcohols, mono, di and triglycerides and their derivatives, and alcohol ethoxylates. Non-limiting examples include: steareth 20, oleth 10, laureth 4, PEG-12 glyceryl dioleate, glycerol stearate, sorbitan oleate, and PPG-9 buteth-12.

Yet another suitable class of surfactants is anionic surfactants. This class includes alkyl and alkyl ether sulfates generally the formula ROSO₃M and RO(C₂H₄O)ₓSO₃M wherein R is alkyl or alkenyl of from about 10 to 20 carbon atoms, x is 1 to about 10 and M is a water soluble cation such as ammonium, sodium, potassium, or triethanolamine cation.

Surfactants are generally added as part of the dye component, however, in some instances it may be desirable to include surfactant as part of the developer as well. When the surfactant is itself as soluble solid, it may be desirable to incorporate some portion thereof as part of the salt component .

Alkalizing Agents

To provide the alkaline conditions desired to promote oxidative dyeing, in addition to the carbonate releasing salts described above, the compositions of this invention may further comprise one or more additional agents that provide an alkalizing effect. Examples of such alkalizing agents include, but are not limited to: ammonium hydroxide, alkali metal hydroxides and alkaline earth metal hydroxides; amines such as for example, alkanolamines, polyalkylene amines, heterocyclic amines; basic amino acids; and the like. Non-limiting examples of alkalizing agents suitable for use herein are: ammonium hydroxide, sodium hydroxide, potassium hydroxide, magnesium hydroxide, calcium hydroxide, urea, ethylamine, dipropyl amine, triethylamine, 1,3-diaminopropane, monoethanolamine, diethanolamine, triethanolamine, dimethylaminoethanol, diethylenetriamine, morpholine, diethylaminoethanol, aminoalkylpropanediol, L-arginine, lysine, oxylysine, and histidine. Preferred alkalizing agents are ammonium and/or sodium hydroxide.

As an alkaline environment favors the degradation of peroxide, it is desirable for the alkalizing agent to be provided as part of the developer and/or salt component. Desirably, the hair coloring compositions of this invention should have a pH of from about 8 to about 11, preferably from about 9 to about 11, and, in at least one embodiment of particular interest, about 9 to about 10.5.

Thickening Agents

Desirably, the hair coloring compositions further comprise at least one thickening agent. Thickening agent is typically included as part of the developer and/or dye components of the subject compositions. Long chain fatty alcohols having up to about 22 carbon atoms in the long fatty chain can be thickener constituents in the compositions of this invention. Non-limiting examples of such fatty alcohols are: lauryl alcohol, oleyl alcohol, myristyl alcohol, stearyl alcohol, and the like. Mixtures of fatty alcohols are also useful and are commercially available from numerous suppliers.

Thickening agents suitable for use herein may also be selected from long chain fatty acids having up to about 22 carbon atoms in the long fatty chain thereof. Non-limiting examples of such long chain fatty acids include: oleic acid, oleic acid, stearic acid, myristic acid, and linoleic acid. Mixtures of fatty acids are also useful and are commercially available from numerous suppliers.

The fatty alcohols and fatty acids described above may be in alkoxylated form. Desirably, such alkoxylates will contain an average of one to three, more particularly one to two alkylene oxide, preferably ethylene oxide units. Preferably such alkylene units are ethylene oxide units

Other thickening agents such as are conventionally used in hair coloring compositions may be present as optional ingredients in the compositions of this invention.

Chelating Agents

The colorant compositions of this invention comprise one or more chelating agents. The term “chelating agent” (or “chelant” or “sequestering agent”) is well known in the art and refers to a molecule or a mixture of different molecules each capable of forming a chelate with a metal ion. A chelate is an inorganic complex in which a compound (chelant) is coordinated to a metal ion at two or more points so that there is a ring of atoms including the metals. Chelants contain two or more electron donor atoms that form coordination bonds with the metal ion.

As used herein, the term “chelant” includes all salts and derivatives comprising the same functional structure as the parent chelant they are referring to that have similar or better chelating properties. The term “derivatives” also includes “chelating surfactant” compounds (these are chelants modified to bear a surfactant moiety while keeping the same chelating functionality, see U.S. Pat. No. 5,284,972, “N-acyl-N,N′,N′-ethylenediaminetriacetic acid” for an example of modified ethylenediaminetriacetic acid). The term “derivatives” also includes large molecules comprising one or more chelating groups having the same functional structure as the partent chelants. An example of these large molecules is polymeric EDDS (ethylenediaminedisuccinic acid).

Preferred chelants for use herein are carboxylic acids (in particular aminocarboxylic acids), phosphonic acids (in particular aminophosphonic acids), and polyphosphoric acids (in particular linear polyphosphoric acids), their salts and derivatives.

Aminocarboxylic Acid Chelants

Carboxylic acid chelants as defined herein are chelants having at least one carboxylic acid moiety (—COOH).

Examples of aminocarboxylic acid chelants suitable for use herein include nitrilotriacetic acid and polyaminocarboxylic acids such as diethylenetriamine pentaacetic acid (DTPA), ethylenediamine disuccinic acid (EDDS), ethylenediamine diglutaric acid (EDGA), 2-hydroxypropylenediamine disuccinic acid (HPDS), glycinamide-N,N′-disuccinic acid (GADS), ethylenediamine-N—N′-diglutaric acid (EDDG), 2-hydroxypropylenediamine-N—N′-disuccinic acid (HPDDS), ethylenediaminetetraacetic acid (EDTA), dipicolinic acid (DPA), salts thereof and derivatives thereof.

Other suitable aminocarboxylic chelants for use herein are iminodiacetic acid derivatives such as N-2-hydroxyethyl N,N diacetic acid or glyceryl imino diacetic acid (described in EP-A-317,542 and EP-A-399,133), iminodiacetic acid-N-2-hydroxypropyl sulfonic acid and aspartic acid N-carboxymethyl N-2-hydroxypropyl-3-sulfonic acid (described in EP-A-516,102), -alanine-N,N′-diacetic acid, aspartic acid-N,N′-diacetic acid, aspartic acid-N-monoacetic acid and iminodisuccinic acid chelants (described in EP-A-509,382), ethanoldiglycine acid, salts thereof and derivatives thereof.

EP-A-0 476 257 describes suitable amino based chelants. EP-A-0 510 331 describes suitable chelants derived from collagen, keratin or casein. EP-A-0 528 859 describes suitable alkyl iminodiacetic acid chelants. Dipicolinic acid and 2-phosphonobutane-1,2,4-tricarboxylic acid are also suitable.

Preferred aminocarboxylic chelants are diamine-N,N′-dipolyacid and monoamine monoamide-N,N′-dipolyacid chelants, salts thereof and derivatives thereof. Preferred polyacids contain at least two acid groups independently selected from the carboxylic acid group (—COOH), sulfonic group (—SO₃H), the o-hydroxyphenyl group, the m-hydroxyphenyl group and the p-hydroxyphenyl group. Suitable polyacids include diacids, triacids and tetraacids, preferably diacids. Preferred salts include alkali metal, alkaline earth, ammonium or substituted ammonium salts. EDTA is a particularly preferred chelant.

Preferably, the polyacids are di-carboxylic acids, preferably di-carboxylic acids having a carbon chain length of from about 3 to about 10 carbon atoms, more preferably from about 4 to about 6 carbon atoms, even more preferably about 4 carbon atoms.

Exemplary diamine dipolyacids suitable for use herein include ethylenediamine-N,N′-disuccinic acid (EDDS), ethylenediamine-N,N′-diglutaric acid (EDDG), 2-hydroxypropylenediamine-N,N′-disuccinic acid (HPDDS), all disclosed in European Pat. EP 0 687 292, ethylenedicysteic acid (EDC) disclosed in U.S. Pat. No. 5,693,854, diaminoalkyldi(sulfosuccinic acids) (DDS) disclosed in U.S. Pat. No. 5,472,642 and EDDHA (ethylenediamine-N-N′-bis(ortho-hydroxyphenyl acetic acid)), a method of preparation of which is disclosed in EP 331,556. A preferred monoamine monoamide-N,N′-dipolyacid is glycinamide-N,N′-disuccinic acid (GADS), described in U.S. Pat. No. 4,983,315.

Highly preferred for use herein is ethylenediamine-N,N′-disuccinic acid (EDDS), derivatives and salts thereof. Preferred EDDS compounds for use herein are the free acid form, and salts thereof. Preferred salts include alkali metal, alkaline earth metals, ammonium and substituted ammonium salts (e.g. monoethanolammonium, diethanolammonium, triethanolammonium). Highly preferred salts are sodium, potassium, magnesium and calcium salts. Examples of such preferred sodium salts of EDDS include Na₂EDDS and Na₃EDDS.

Preferred aminocarboxylic acid chelants that are not diamine-N,N′-dipolyacid and monoamine monoamide-N,N′-dipolyacid chelants include N,N′-bis(2-hydroxybenzyl)ethylenediamine-N,N′-diacetic acid (HBED) salts thereof and derivatives thereof.

Polyphosphoric Acid Chelants

Suitable polyphosphoric acid type chelants include molecules that contain more than one P atom and have P—O—P bonds. Polyphosphoric acid chelants and salts (polyphosphates) can be linear and are generally represented by the formula [PₙO₃ₙ₊₁]⁽ⁿ⁺²⁾⁻M₍ₙ₊₂)⁺ wherein M is a suitable counter-ion such as H⁺, Na⁺ or K⁺ and n an integer. Polyphosphoric acid type chelants and their polyphosphate salts can also be cyclic and have the formula [PₙO₃ₙ]ⁿ⁻Mₙ⁺. Representative examples include, among others, sodium tripolyphosphate, tetrasodium diphosphates, hexametaphosphoric acid and sodium metaphosphate.

Phosphonic Acid Chelants

Suitable phosphonic acid type chelants include amino alkylene poly (alkylene phosphonic acid), ethane 1-hydroxy diphosphonic acids and nitrilo trimethylene phosphonic acids, salts thereof and derivatives thereof. Suitable chelants of this type are disclosed in U.S. Pat. No. 4,138,478, Reese et al., U.S. Pat. No. 3,202,579 and U.S. Pat. No. 3,542,918, Berth et al., all incorporated herein by reference.

Preferred chelants for use herein are aminotri-(1-ethylphosphonic acid), ethylenediaminetetra-(1-ethylphosphonic acid), aminotri-(1-propylphosphonic acid), and aminotri-(isopropylphosphonic acid).

Especially preferred chelants are aminotri-(methylenephosphonic acid), ethylene-diamine-tetra-(methylenephosphonic acid) (EDTMP) and diethylene-triamine-penta(methylenephosphonic acid) (DTPMP).

For ease of formulation, the chelant is desirably present in the salt component (c), particularly as the amounts of interest may exceed the solubility limitations of the other system components. Chelant may also be present in the dye and/or developer components, provided that it does not undesirably react with these components.

Chelant is present in the compositions of this invention in an amount of at least about 1% by weight, more particularly from about 1 to about 5% by weight, and preferably from about 1 to about 3% by weight, based on the combined weight of the dye, developer and salt components thereof.

Oxidizing Agents

At least one peroxide-releasing compound is present in the developer component of the subject compositions as an oxidizing agent. Normally, the peroxide-releasing compound is hydrogen peroxide or a source which generates this material or a hydroperoxyl radical, e.g., the ammonium, alkali and alkaline earth metal persulfates and perborates.

In order to stabilize the hydrogen peroxide that is present, the developer component will normally be formulated to have a pH ranging from about 2 to about 5, preferably from 2.5 to 3.5. One or more buffering agents may be included within the developer component to assist in maintaining a desired pH. Amounts of the buffering agents may from range from about 0.001 to about 2%, preferably from about 0.01 to about 0.1% by weight of the developer component. Phosphoric, hydrochloric, sulfonic and C₂-C₃₀ carboxylic acids and their salts are useful as buffering agents. Illustrative examples of buffering agents include but are not limited to: tartaric acid, citric acid, acetic acid, lactic acid, ammonium sulfate, sodium dihydrogen phosphate/phosphoric acid, potassium chloride/hydrochloric acid, potassium dihydrogen phthalate/hydrochloric acid, sodium citrate/hydrochloric acid, potassium dihydrogen citrate/hydrochloric acid, sodium tartarate/tartaric acid, sodium lactate/lactic acid, sodium acetate/acetic acid, disodium hydrogen phosphate/citric acid and sodium chloride/glycine/hydrochloric acid

The weight ratio of dye component to developer component typically ranges from about 1:3 to about 1:0.5, depending upon the particular formulation of the particular dye, developer and salt components employed. In the practice of this invention such ratios are commonly from about 1 to about 2.

Optional Ingredients

The colorant compositions of the present invention can comprise a wide range of optional ingredients such as are conventionally used in colorant compositions based on oxidative dyes. Non-limiting examples of these functional classes include: antioxidants, conditioning agents, viscosity modifiers, emulsifiers, fragrance components, preservatives, and the like.

In a hair coloring treatment according to the method of this invention, just prior to application the dye, developer and salt components are mixed together to form a hair coloring composition. The resulting hair colorant composition is then applied to the hair, typically within a period that does not exceed 45 minutes after mixing. Optionally, the hair can be made wet prior to application of the coloring composition; preferably it is dry. Application temperatures are typically in the range of from about 15° C. to about 40° C. The colorant composition is allowed to remain on the hair for a period of from about 2 minutes to about 60 minutes, more particularly from about 5 minutes to about 30 minutes, depending upon the degree of coloration desired. The hair is then rinsed, preferably with water and/or a neutralizing agent. If desired, the hair may be subjected to one or more post-coloring conditioning treatments.

Kit Containing an Instruction Sheet

The present invention also relates to a kit for carrying out the hair coloring method of this invention. The kit comprises dye, developer, and salt as individually packaged components. By “individually packaged” it is meant that the components are contained in containers or packaging that allows them to be kept apart until just before use. The kit also includes written instructions that explain how the hair coloring compositions of the invention are prepared and used. Optionally, the kit may further comprise one or more post coloring treatment compositions such as for example, neutralizing solutions, shampoos and/or conditioners. Optionally, the kit may further comprise an additional optional component a mixing vessel for preparation of the hair coloring composition. Alternatively, the packaging for the dye, developer or salt component may be sized to accommodate the combination and mixing of the hair coloring composition components.

As used herein, the term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

Except in the operating and comparative examples, or where otherwise explicitly indicated, all numbers in this description indicating amounts of material, including material ratios, ought to be understood as modified by the word “about”.

### EXAMPLES

The following examples will more fully illustrate the embodiments of this invention. All parts, percentages and proportions referred to here and elsewhere in the specification and the appended claims are by weight, unless specified otherwise.

### Example 1

This Example illustrates the lightening or lifting ability of a hydrogen peroxide-based bleaching composition where a separate salt component is used to provide the composition with relatively high levels of carbonate-releasing salt. Parts A1, B and C of the Example 1 compositions were prepared according to the formulations described in Tables 1A through 1C.

TABLE 1A
Part A1

| Ingredient | Weight % |
|---|---|
| Sodium Sulfite (Anhydrous) | 0.80 |
| Erythorbic Acid, USP | 0.15 |
| Versene, powder (tetrasodium EDTA) | 0.10 |
| Propylene glycol, USP, 100% | 7.00 |
| Isopropyl Alcohol (99%) | 11.00 |
| Oleic Acid | 5.00 |
| Alcohol, C12-C15, Ethoxylated Pareth 9 | 16.00 |
| (Tomadol 2) | |
| Alcohol, C12-C15, Ethoxylated Pareth 3 | 16.00 |
| Ammonium Hydroxide, 28% | 4.00 |
| Rose Delight UN 137986 | 0.50 |
| Deionized Water | To 100 |

TABLE 1B
Part B
(Developer Component)

| | | Weight % |
|---|---|---|
| Ingredient | % active | (as supplied) |
| Oleth 10 | 100 | 0.83 |
| Stearth-21 | 100 | 0.95 |
| Oleyl Alcohol | 100 | 0.83 |
| Cetyl Alcohol | 100 | 2.49 |
| Dicetyldimonium Choloride/ | 68/27 | 1.66 |
| Propylene Gycol | | |
| Polyquaternium 37 | 48 | 0.83 |
| Pentasodium Pentate | 40 | 0.05 |
| Hydrogen Peroxide | 50 | 18.00 |
| Sodium Acid Pyrophosphate | 100 | 0.05 |
| (Granular) | | |
| Phosphoric Acid (85%) | 85 | 0.045 |
| Sodium Stannate | 100 | 0.02 |
| Deionized Water | | To 100 |

TABLE 1C
Part C
(Salt Component)

| Ingredient | Weight % |
|---|---|
| Disodium EDTA | 10 |
| Texapon K1296 | 2 |
| Ammonium Carbonate | 40 |
| Sodium Carbonate | 48 |

Bleaching compositions were prepared by mixing Parts A1, B and C in the amounts set forth in Table I D.

TABLE 1D
Bleaching Compositions

| | Part A1 | Part B | Part C |
|---|---|---|---|
| Hair Coloring Composition | wt (g) | Wt (g) | wt (g) |
| Control 1 - No Carbonate | 40 | 80 | — |
| Control 2 - 6% Total Carbonate | 40 | 80 | 8.6 |
| Composition 1 - 13% Total Carbonate | 40 | 80 | 20 |
| Composition 2 - 17% Total Carbonate | 40 | 80 | 28 |

The lightening ability of the Table 1D bleaching compositions was evaluated using either dark or medium brown Caucasian hair tresses. Each tress was about 15 to about 20 cm in length and weighed about 5 g. For each bleaching composition tested, 2 tresses were used; the two tresses were treated with approximately 10 g each of the bleaching compositions to be tested. The bleaching composition was allowed to remain on the tresses for 45 minutes and then was removed by rinsing the tresses in water. The tresses were dried and then measured for changes in color intensity (dark to light) using a Hunter Colorimeter (Labscan XE Model), and the resulting Hunter ΔL values reported as an average for the 2 tresses. The procedure was repeated, with the tresses being subjected to a maximum of 5 bleaching treatments. Hunter ΔL values (average for two tresses) after 1, 3 and 5 bleaching treatments are reported in Table 1 E below.

TABLE 1E
Lightening Results

Hunter ΔL values

| | 1 treatment | 3 treatments | 5 treatments |
|---|---|---|---|
| Dark Brown | | | |
| Hair | | | |
| Control 1 | 2.0 | 7.0 | 10.2 |
| Control 2 | 3.8 | 8.7 | — |
| Composition 1 | 10.4 | 20.6 | 23.4 |
| Composition 2 | 12.2 | 22.5 | 25.3 |
| Medium Brown | | | |
| Hair | | | |
| Control 1 | 3.1 | 7.4 | 11.6 |
| Composition 1 | 11.3 | 26.6 | 26.57 |
| Composition 2 | 12.7 | 27.7 | 27.7 |

As demonstrated by the Table 1E data, the high carbonate compositions of this invention were significantly more particularly effective in lightening compared to both the control composition that lacked carbonate as well as the control composition that contained carbonate at a level of 6%.

### Example 2

This Example illustrates the effect of ammonium carbonate level on color deposition. In this Example, Part A2 (dye component) was prepared according to the formulation described in Table 2A, and Part B (developer component) and Part C (salt component) were as described in Tables 1B, and 1C of Example 1. Hair coloring compositions were prepared by mixing components these components in the amounts described in Table 2B.

TABLE 2A
Part A2
(Dye Component)

| Ingredient | Weight % |
|---|---|
| Edenor TiO₅ | 5.00 |
| Tomadol 25-3 | 16.00 |
| Tomadol 25-9 | 16.00 |
| Propylene glycol, USP, 100% | 7.00 |
| Isopropyl Alcohol (99%) | 11.00 |
| Delight 019 | 0.50 |
| EDTA | 0.10 |
| Sodium Sulfite | 0.80 |
| Erythorbic Acid | 0.15 |
| Rodol POAC | 1.12 |
| Rodol P base | 0.29 |
| Rodol D Type J | 0.84 |
| Rodol RS Tech | 0.01 |
| Rodol PAOX | 0.58 |
| Ammonium Hydroxide | 6.00 |
| Deionized Water | To 100 |

TABLE 2B
Hair Coloring Compositions

| | Part A2 | Part B | Part C |
|---|---|---|---|
| Hair Coloring Composition | wt (g) | wt (g) | wt (g) |
| Control 3 - (No Carbonate) | 40 | 80 | — |
| Control 4 - (6% Total Carbonate) | 40 | 80 | 8.6 |
| Composition 3 - (13% Total Carbonate) | 40 | 80 | 20 |

Color deposition of the Table 2B hair coloring compositions was evaluated on dark brown Caucasian hair tresses that were treated and tested following the procedures and using the equipment described in Example 1, except that in this Example changes in Hunter a values were measured, and these values were measured after only one treatment. The color deposition data is reported in Table 2C (average for 2 tresses).

TABLE 2C
Color Deposition Results

| Dark Brown Hair | Hunter Δa values |
|---|---|
| Control 3 | 2.9 |
| Control 4 | 3.5 |
| Composition 3 | 6.5 |

As demonstrated by the Table 2C data, the high carbonate composition in accordance with this invention was significantly more effective in depositing color compared to both the control composition that lacked carbonate as well as the control composition that contained carbonate at a level of 6%.

What is claimed is:

1. A permanent hair coloring composition comprising the following individual components which are combined just prior to application to the hair:
(a) a dye component comprising oxidation dye precursor,
(b) a developer component comprising peroxide-releasing compound, and
(c) a salt component comprising carbonate-releasing salt,
wherein the dye, developer and salt components are formulated to provide the hair coloring composition with: (i) at least one ammonium carbonate salt and at least one additional soluble carbonate salt other than an ammonium carbonate salt, wherein the ratio, by weight, of ammonium carbonate salt to additional soluble carbonate salt is from about 1:0.6 to about 1:1.6, (ii) a total soluble carbonate salt content of greater than about 10% by weight, based on the combined weight of the dye, developer and salt components, and (iii) at least about 1% by weight of chelating agent, based on the combined weight of the dye, developer and salt components.

2. A permanent hair coloring composition as described in claim 1 wherein the total soluble carbonate salt content is from about 12% to about 25% by weight, based on the combined weight of the dye, developer and salt components.

3. A permanent hair coloring composition as described in claim 1 wherein the chelating agent is present in an amount of from about 1 to about 5% by weight, based on the combined weight of the dye, developer and salt components.

4. A permanent hair coloring composition as described in claim 1 which has a pH of from about 9 to about 11 when the individual components have been combined.

5. A permanent hair coloring composition as described in claim 1 wherein the ammonium carbonate salt is selected from the group consisting of ammonium carbonate, ammonium bicarbonate and ammonium carbamate.

6. A permanent hair coloring composition as described in claim 1 wherein the ammonium carbonate salt comprises ammonium carbonate.

7. A permanent hair coloring composition as described in claim 1 wherein the additional carbonate salt comprises alkali metal carbonate.

8. A permanent hair coloring composition as described in claim 1 wherein the salt component is in particulate form.

9. A permanent hair coloring composition as described in claim 1 wherein the peroxide-releasing compound comprises hydrogen peroxide.

10. A kit comprising the following individually packaged components:
(a) a dye component comprising oxidation dye precursor,
(b) a developer component comprising at least one peroxide-releasing compound, and
(c) a salt component comprising carbonate-releasing salt,
wherein the kit further comprises directions instructing that the dye, developer, and salt components are combined just prior to use to form a hair coloring composition, and wherein said components are formulated to provide the resulting hair coloring composition with: (i) at least one ammonium carbonate salt and at least one additional soluble carbonate salt other than an ammonium carbonate salt, wherein the ratio, by weight, of ammonium carbonate salt to additional soluble carbonate salt is from about 1:0.6 to about 1:1.6, (ii) a total soluble carbonate salt content of greater than about 10% by weight, based on the combined weight of the dye, developer and salt components, IS and (iii) at least about I % by weight of chelating agent, based on the combined weight of the dye, developer and salt components.

11. A kit as described in claim 10 wherein the salt component is in powder, particle, granule, pellet, tablet or other solid form.

12. A kit as described in the claim 10 wherein the chelating agent is present in an amount of from about 1 to about 5% by weight, based on the combined weight of the dye, developer and salt components.

13. A kit as described in claim 10 wherein the salt component comprises ammonium carbonate and sodium carbonate.

14. A kit as described in claim 10 wherein the total soluble carbonate salt content is from about 13% to about 20% by weight, based on the combined weight of the dye, developer and salt components

15. A kit as described in claim 10 wherein the dye and developer components further comprise cosmetically acceptable carrier.

16. A kit as described in claim 10, wherein the ratio, by weight, of ammonium carbamate salt to additional soluble carbamate salt is from about 1.0:0.7 to about 1.0:1.4.

17. A kit as described in claim 10 wherein the dye component further comprises surfactant.

18. A kit as described in claim 10 which is a box colorant.

19. A method for lightening hair that comprises the steps of
(1) forming a hair coloring composition by mixing together the following individually packaged components:
(a) a dye component comprising oxidation dye precursor,
(b) a developer component comprising peroxide-releasing compound, and
(c) a salt component comprising carbonate-releasing salt, wherein the dye, developer and salt components are formulated to provide the hair coloring composition with: (i) at least one ammonium carbonate salt and at least one additional soluble carbonate salt other than an ammonium carbonate salt, wherein the ratio, by weight, of ammonium carbonate salt to additional soluble carbonate salt is from about 1:0.6 to about 1:1.6, (ii) a total soluble carbonate salt content of greater than about 10% by weight, based on the combined weight of the dye, developer and salt components, and (iii) at least about I % by weight of chelating agent, based on the combined weight of the dye, developer and salt components,
(2) applying the hair coloring composition to the hair,
(3) allowing the hair coloring composition to remain on the hair for a period sufficient to achieve a desired degree of coloration and
(4) rinsing the hair.
